(19) United States
(12) Patent Application Publication
(10) Pub. No.: US 2009/0070044 A1
(43) Pub. Date: Mar. 12, 2009

(54) Two-Body Interaction Calculation Circuit and Many-Body Problem Calculation Device that Uses this Circuit

(76) Inventor: Kazuhiko Watanabe, Tokyo (JP)

Correspondence Address: NEC CORPORATION OF AMERICA, 6535 N. STATE HWY 161, IRVING, TX, 75039, US

(21) Appl. No.: 12/048,025

(22) Filed: Mar. 13, 2008

(30) Foreign Application Priority Data
Mar. 14, 2007 (JP) 2007-064783

(51) Int. Cl. G01N 33/48

(52) U.S. Cl. 702/19

(57) ABSTRACT

The object of the present invention is to provide both a two-body interaction calculation circuit that can reduce the number of arithmetic elements of a circuit for virial calculation and that can decrease circuit scale and a many-body problem calculation device that uses this two-body interaction calculation circuit, the two-body interaction calculation circuit of the present invention including: a coordinate difference calculation unit for receiving as input coordinates of center particles i, the size of a basic cell, lattice vectors, and coordinates of particles j and for calculating the difference between coordinates of a center particle i and coordinates in which the coordinates of a particle j have been converted to a mirror-image particle; a distance-squaring calculation unit for calculating the square of distances based on the difference; a function calculation unit for calculating an interaction function that depends on distance based on the square of distance; a force calculation unit for calculating force from the interaction function and the difference; a sum-total calculation unit for calculating the sum-total of force for particles i based on force; and a virial calculation unit for calculating virials based on the sum-total of force.

This application is based upon the claims and benefit of priority from Japanese patent application No. 2007-064783, filed on Mar. 14, 2007, the disclosure of which is incorporated herein in its entirety by reference.

### BACKGROUND OF THE INVENTION

1. Field of the Invention

The present invention relates to a many-body problem calculation device.

2. Description of the Related Art

In the field of molecular dynamics, the behavior of liquids, solids, and macromolecules is thought to be the result of the movement of the atoms or molecules that make up these liquids, solids, and macromolecules, and investigation is being conducted through simulation of the movement of these particles. The problem of treating systems made up of a multiplicity of particles that interact with each other is referred to as the many-body problem, and in molecular dynamics, many-body problem calculations are carried out in which atoms and molecules are treated as particles.

Because interaction takes place between particles of a system treated by molecular dynamics, the force that works between particles is an important physical quantity in molecular dynamics and must be calculated. The force that acts upon a particular particle is the sum total of the force exerted from all other particles.

The pressure and volume of mass changes with changes in temperature, and because change in volume means the position of a particle changes, pressure is also an important physical quantity. Virials used for finding this pressure are also frequently calculated as an important physical quantity. A virial is obtained by multiplying the force that works upon particles by the particle coordinates and then taking the sum of products. Pressure can be found from the virial of a system.

Patent document 1 (JP-A-H08-287042) is cited as one invention of a virial calculation circuit.

The pressure used in the calculations of molecular dynamics is calculated from the virial, temperature, and volume of a system. In periodic boundary conditions in which the borders of a basic cell indicated by the lattice vector n=(0, 0, 0) are surrounded by image cells indicated by n=(nₓ, n_{y}, n_{z}) as shown in FIG. 1, a virial calculation is carried out by the calculation formula shown by Equation (1).

$${V}_{xx} = \frac{1}{2} {\sum }_{i} {\sum }_{j} ( {x}_{i} - {x}_{j} ) {f}_{xij} + \frac{1}{2} {\sum }_{i} {{\sum }_{n}}^{′} {{\sum }_{j}}_{xi} \{ {x}_{i} - ( {x}_{j} + {n}_{x} {L}_{x} ) \} {f}_{xijn} \qquad ( 1 )$$

In recent years, there has been a growing demand in molecular dynamics simulations for long-term simulation of a large-scale protein, and a high-speed many-body problem calculation device is desired for this purpose. For high speeds, a means for increasing the degree of parallel operations of a pipeline for calculating force and virials is typical, but improvement of performance and improvement of the degree of parallel operations of a pipeline come with a proportional increase in the amount of hardware, and improvement in performance therefore is accompanied by increases in cost and power consumption. Accordingly, a method for constructing a calculation pipeline with less hardware is crucial for achieving lower cost and lower power consumption.

A method in which the forces between particles and the distances between particles are multiplied, or a method in which the sum total of the forces between particles in image cells is multiplied by coordinates that have undergone mirror-image conversion have been proposed as methods of calculating virials in molecular dynamics calculations that use a many-body problem calculation device, but both methods required adders and multipliers as the circuit for calculating virials.

In typical molecular dynamics calculations, numerical calculations are carried out by floating points, and sum total calculations in particular require at least double precision accuracy. In addition, methods for increasing pipelines and increasing the amount of parallel operations have been adopted for achieving calculations at higher speed, but such methods are problematic in that floating points computing elements entail a large amount of hardware, resulting in a massive amount of hardware as a whole and the consumption of a large amount of power.

### SUMMARY OF THE INVENTION

It is an object of the present invention to provide both a two-body interaction calculation circuit that enables a decrease of the number of computing element circuits that perform virial calculations and a reduction of circuit scale and a many-body problem calculation device that uses this two-body interaction calculation circuit.

The two-body interaction calculation circuit of the present invention includes:

a center particle coordinate holding unit for holding coordinates of center particles i;
a basic cell information holding unit for holding the size of a basic cell;
a lattice vector holding unit for holding lattice vectors;
a coordinate difference calculation unit for receiving as input coordinates of center particles i that are held by the center particle coordinate holding unit, the size of a basic cell that is held by the basic cell information holding unit, lattice vectors that are held by the lattice vector holding unit, and coordinates of particles j, and for calculating the difference between the coordinates of center particles i and coordinates in which coordinates of particles j have been converted to mirror particles;
a distance-squaring calculation unit for calculating the square of distance from differences found in the coordinate difference calculation unit;
a function calculation unit for calculating a function of interaction according to distance based on the square of distance found in the distance squaring calculation unit;
a force calculation unit for calculating force from the difference found in the coordinate difference delay unit and the interaction function found in the function calculation unit;
a sum-total calculation unit for calculating a sum total of force for particles i based on force found in the force calculation unit; and
a virial calculation unit for calculating virials from the sum total of force found in the sum-total calculation unit.

The many-body problem calculation device of the present invention is composed of:

the above-described two-body interaction calculation circuit; and
a computer system for obtaining the sum total of the force for each particle i and the sum total of the virials for all particles i from the two-body interaction calculation circuit and calculating the entire virial.

### BRIEF DESCRIPTION OF THE DRAWINGS

FIG. 1 shows the state in which virial calculation is carried out;

FIG. 2 shows the state when the lattice vectors shown in FIG. 1 are viewed in the x-y plane with a lattice vector of the z-axis as “0”;

FIG. 3 is a block diagram showing the overall configuration of the first embodiment of a two-body interaction calculation circuit;

FIG. 4 is a circuit diagram showing the actual configuration of force calculation unit 308, sum total calculation unit 309, and virial calculation unit 310 that are shown in FIG. 3, and

FIG. 5 is a flow chart showing the operation of a many-body problem calculation device according to the present invention.

### EXEMPLARY EMBODIMENT

Explanation next regards an embodiment of the present invention with reference to the accompanying figures.

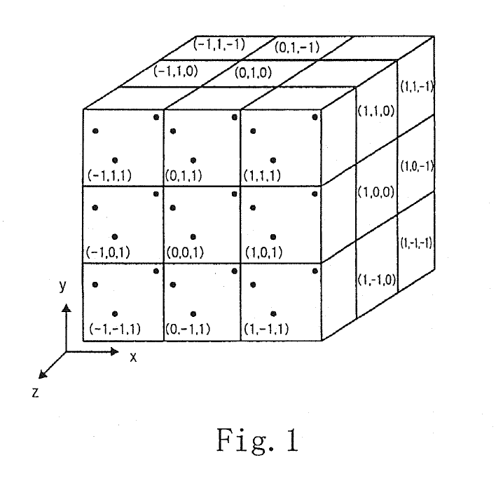

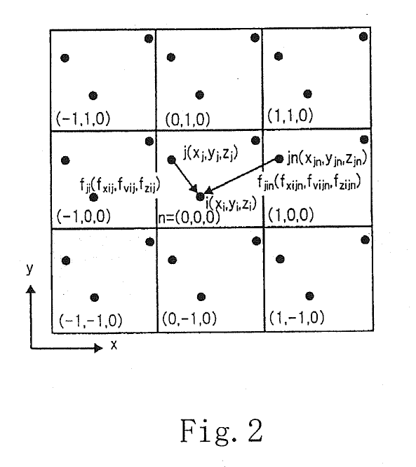

Explanation first regards the basic concept of the present invention. FIG. 2 shows the state of lattice vectors shown in FIG. 1 as viewed in the xy-plane with a lattice vector of the z-axis as “0.”

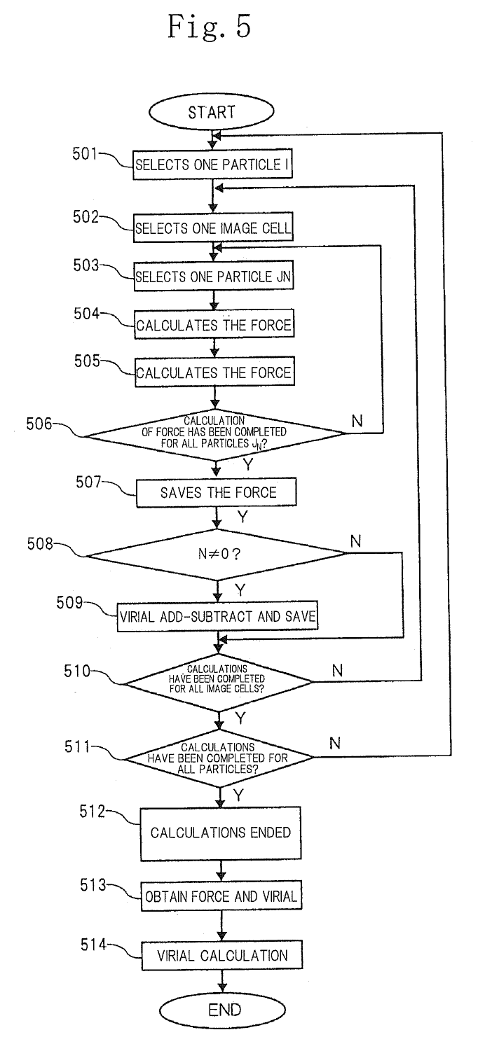

In FIG. 2, in a basic cell represented by lattice vector n=(0, 0, 0) in which the length of each axis of the xyz coordinate axes is Lₓ, L_{y}, and L_{z}, the position of a particular center particle i is represented by (xᵢ, yᵢ, zᵢ) and the position of particle j other than particle i in the basic cell is represented by (xⱼ, yⱼ, zⱼ); in image cell n represented by the lattice vector n=(nₓ, n_{y}, n_{z}), particle in is represented by coordinates (xⱼₙ, yⱼₙ, zⱼₙ); the x-coordinate component of force fᵢⱼ that is exerted upon particle i from particle j is fₓᵢⱼ; and the x-coordinate component of force fᵢⱼₙ that is exerted from particle jₙ is fₓᵢⱼₙ; virial Vxx of the xx-component of the pressure tensor is represented by Equation (1). In addition, Σ′ of the second term on the right of Equation (1) indicates that the basic cell n=(0, 0, 0) is not contained in n. Focusing now on Equation (2), the first term on the right is the sum of the products for all particles obtained by multiplying the sum total of forces Fₓᵢ that are exerted upon particle i from the other particle j calculated by 308 and 309 of FIG. 5 according to the x-coordinate Xᵢ of particle i, and because the amount of calculations for multiplication and summing of products is on the order of O(N), calculations can be realized by software without necessitating calculation by hardware. One-half and the length Lₓ of the x-axis of the basic cell of the second term on the right of Equation (2) are constants, and the multiplication may be calculated by software.

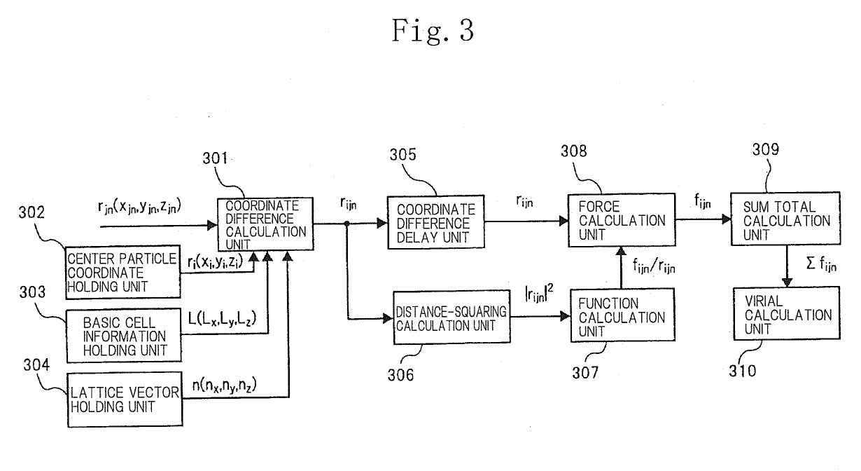

FIG. 3 is a block diagram showing the overall configuration of an embodiment of the two-body interaction calculation circuit according to the present invention.

The present embodiment is made up of: center particle coordinate holding unit 302, basic cell information holding unit 303, lattice vector holding unit 304, coordinate difference calculation unit 301, coordinate difference delay unit 305, distance-squaring calculation unit 306, function calculation unit 307, force calculation unit 308, sum total calculation unit 309, and virial calculation unit 310.

Center particle coordinate holding unit 302 holds the coordinates rᵢ(xᵢ, yᵢ, zᵢ) of particle i, basic cell information holding unit 303 holds the size L (Lₓ, L_{y}, L_{z}) of the basic cell, and lattice vector holding unit 304 holds lattice vector n (nₓ, n_{y}, n_{z}). Coordinate difference calculation unit 301 takes as input the coordinates rᵢ(xᵢ, yᵢ, zᵢ) of center particle i that are held by center particle coordinate holding unit 302, the size of L (Lx, Ly, Lz) of the basic cell that is held by basic cell information holding unit 303, lattice vector n (nₓ, n_{y}, n_{z}) that is held by lattice vector holding unit 304, and coordinates rⱼₙ(xⱼₙ, yⱼₙ, zⱼₙ) of particle j, and calculates the difference rᵢⱼₙ=(xᵢ−(xⱼₙ+nₓLₓ) yᵢ−(yⱼₙ+n_{y}L_{y}), zᵢ−(zⱼₙ+n_{z}L_{z})) of coordinates rᵢ of center particle i and rⱼₙ′=(xⱼₙ+nₓLₓ, yⱼₙ+n_{y}L_{y}, zⱼₙ+n_{z}L_{z}) that are the coordinates rⱼₙ of particle j converted to a mirror-image particle.

Coordinate difference delay unit 305 holds rᵢⱼₙ that is found in coordinate difference calculation unit 301 and supplies output to force calculation unit 308. Distance-squaring calculation unit 306 calculates the square of the distance |rᵢⱼₙ|² from rᵢⱼₙ that was found in coordinate difference calculation unit 301. Function calculation unit 307 calculates g(r)=fᵢⱼₙ/rᵢⱼₙ in accordance with the interaction function g(r) that depends on distance r according to squared distance |rᵢⱼₙ|² found in distance-squaring calculation unit 306.

Force calculation unit 308 calculates force fᵢⱼₙ=(fₓᵢⱼₙ, f_{yijn, f}_{zijn}) from fᵢⱼₙ/rᵢⱼₙ found in function calculation unit 307 and rᵢⱼₙ from coordinate difference delay unit 305. Sum-total calculation unit 309 calculates the sum total of force Σfᵢⱼₙ for particle i from force fᵢⱼₙ found in force calculation unit 308. Virial calculation unit 310 calculates the virial from the sum total of force Σfᵢⱼₙ found in sum-total calculation unit 309. In the circuit shown in FIG. 3, coordinates rᵢ(xᵢ, yᵢ, zᵢ) of particle i that are held in center particle coordinate holding unit 302, basic-cell size L (Lₓ, L_{y}, L_{z}) held in basic cell information holding unit 303, and lattice vector n (nₓ, n_{y}, n_{z}) held in lattice vector holding unit 304 are set in advance by a means not shown in the figure.

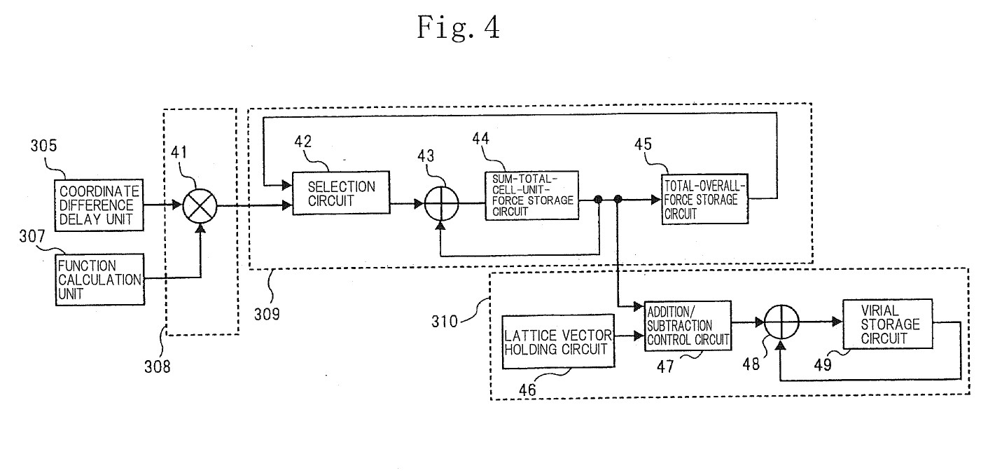

FIG. 4 is a circuit diagram showing the actual configuration of force calculation unit 308, sum total calculation unit 309, and virial calculation unit 310 of FIG. 3. Although there are properly three circuits for the x-coordinate component, the y-coordinate component, and the z-coordinate component, only the circuit configuration for the x-coordinate component is shown because the circuit configurations of all of these circuits are identical.

In the circuit shown in FIG. 4, force calculation unit 308 is made up from multiplier 41 and calculates the x-coordinate component fₓᵢⱼₙ of the force that acts between particles i and j by multiplying by multiplier 41 rₓᵢⱼₙ supplied as output from coordinate difference delay unit 305 and g(r)=fᵢⱼₙ/rᵢⱼₙ supplied as output from function calculation unit 307, and supplies this result to sum total calculation unit 309.

Sum-total calculation unit 309 is made up by: selection circuit 42, adder 43, sum-total-cell-unit-force storage circuit 44, and sum-total-overall-force storage circuit 45. Output fₓᵢⱼₙ of force calculation unit 308 is selected by selection circuit 42 and supplied to adder 43. Adder 43 supplies the result Σfₓᵢⱼₙ of adding the sum total of force Σfₓᵢⱼ₋₁ₙ, that works upon particle i up to particle j−1 that is saved in sum-total-cell-unit-force storage circuit 44 and output fₓᵢⱼₙ of force calculation unit 308, to sum-total-cell-unit-force storage circuit 44, and Σfₓᵢⱼₙ is held in sum-total-cell-unit-force storage circuit 44.

Upon completion of addition for all particles in jₙ one cell, selection circuit 42 selects the sum total of force Σfₓᵢⱼₙ₋₁ up to cell n−1 that is held in sum-total-overall-force storage circuit 45 of particle i and supplies this value to adder 43. Adder 43 adds Σfₓᵢⱼₙ that is held in sum-total-cell-unit-force storage circuit 44 to sum-total force ΣΣfₓᵢⱼₙ₋₁ and supplies the result as ΣΣfₓᵢⱼₙ to total-overall-force storage circuit 45 of particle i, and sum-total-overall-force storage circuit 45 holds ΣΣfₓᵢⱼₙ. At the same time, the output of sum-total-cell-unit-force storage circuit 44 is supplied to virial calculation unit 310.

Virial calculation unit 310 is made up by lattice vector holding circuit 46, addition/subtraction control circuit 47, adder 48, and virial storage circuit 49, and lattice vector n (nₓ, n_{y}, n_{z}) is set in advance in lattice vector holding circuit 46 by a means not shown in the figure.

Addition/subtraction control circuit 47 changes the value of the result Σfₓᵢⱼₙ of addition for all particles jn in one cell that is the output of sum-total calculation unit 309 in accordance with lattice vector nₓ that is held in cell information holding circuit 46, and therefore supplies “0” as output if nₓ=0, supplies Σfₓᵢⱼₙ without making any changes if nₓ=1, and inverts the symbol and supplies −Σfₓᵢⱼₙ if nₓ=−1.

Adder 48 adds the output of addition/subtraction control circuit 47 and sum-total virial Vₓₓₙ₋₁ for cells up to n−1 that are held in virial storage circuit 49, supplies Vₓₓₙ as output, and holds the result in virial storage circuit 49.

As previously described, ΣΣnₓΣfₓᵢⱼₙ calculated in virial calculation unit 310 is a value obtained by adding in adder 48 the sum of force exerted from particles jn in image cells n upon particle i that is calculated by force calculation unit 308 and sum-total calculation unit 309 by inverting the symbol, leaving the symbol unchanged, or changing the value to 0 in addition/subtraction control circuit 47 according to the lattice vectors that indicate the positions of image cells that are held in lattice vector holding circuit 46 and that obtain the sum of products for all particles i in virial storage circuit 49. This shows that virials can be calculated by merely adding to a circuit for calculating force, such as force calculation unit 308 and sum-total calculation unit 309, a means for holding the lattice vectors, an adder circuit, and a circuit for manipulating the symbol of the sum-total for each image cell of virial calculation unit 310.

The two-body interaction calculation circuit of the configuration described hereinabove is a device that makes up a many-body problem calculation device for carrying out virial calculation in conjunction with a computer system for carrying out operations by means of software wherein the computer system implements calculation of virials using the sum total of virials obtained by means of a two-body interaction calculation circuit.

Explanation next regards the operation of the many-body problem calculation device according to the present invention with reference to the flow chart shown in FIG. 1.

Selection circuit 42 first selects one particle i that is the object of calculation (Step 501). Selection circuit 42 next selects one image cell n for calculating the force exerted upon particle i (Step 502). Selection circuit 42 next selects one particle jn in this image cell (Step 503).

Force calculation unit 308 then calculates the force exerted upon selected particle i from selected particle jn (Step 504), and sum-total calculation unit 309 sums up the calculation results (Step 505).

Selection circuit 42 here determines whether the calculation of force has been completed for all particles jₙ in the selected image cell (Step 506). If the calculation of all particles jₙ is not completed, selection circuit 42 returns to Step 503 to select the next particle jₙ.

After calculation of all particles in has been completed, the many-body problem calculation device saves the force summation results (Step 507).

Next, for the value of lattice vector n of an image cell, addition/subtraction control circuit 47 determines element nₓ of n when calculating the x-coordinate component, determines element n_{y} of n when calculating the y-coordinate component, and determines element n_{z} of n when calculating the z-coordinate component (Step 508). If the value of n in the determination of each coordinate component is 0, the process advances to Step 510. If the value of n is −1, the symbol of the summation of force is inverted, the virial is summed up, and the summation result is held. If the value of n is 1, the summation of force proceeds as is, the virial is summed up, and virial storage circuit 48 saves the summation results (Step 509).

Selection circuit 42 here determines whether calculations have been completed or not for all image cells in the system (Step 510). If the calculation of virials has not been completed for all image cells, selection circuit 42 returns to Step 502 and selects the next image cell.

If the calculation of virials has been completed for all image cells, selection circuit 42 determines whether calculation of virials has been completed for all particles i (Step 511). If the calculation of virials has not been completed for all particles i, selection circuit 42 returns to Step 501 to select the next particle i. If the calculation of virials has been completed for all particles i, the two-body interaction calculation circuit terminates processing (Step 512).

If processing by two-body interaction calculation circuit has ended, the computer system obtains the sum-total of virials for all particles i and the sum-total of force for each particle i from the two-body interaction calculation circuit (Step 513).

The virial of the entire system is next calculated by software from the force and virials that have been obtained from the two-body interaction calculation circuit, and all processing is terminated (Step 514).

Explanation in the above-described embodiment is in regard to a case in which one layer of image cells surrounds the borders of a basic cell, i.e., a case in which lattice vectors assume values of −1, 0, and 1; but in virial calculation unit 310 shown in FIG. 3, virials can also be calculated for a case in which two or three layers of image cells surround the borders of the basic cell, i.e., a case in which lattice vectors assume values such as ±2 and ±3, by enabling selection of the output of adder 48 and the output of virial storage circuit 49 and then repeating the addition of the output of adder 48 and the output of addition/subtraction control circuit 47 according to the values that have been set in lattice vector holding circuit 46.

Pressure that is used in molecular dynamics calculations is calculated from the, temperature, volume, and virial of the system. The present invention converts the virial calculation formula shown by Equation (1) in periodic boundary conditions in which image cells indicated by n=(nₓ, n_{y}, n_{z}) surround the borders of a basic cell indicated by lattice vector n=(0, 0, 0) as shown in FIG. 2 such that, as shown in Equation (2), the second (0, 0, 0) term on the right for which the order of the amount of calculations is O(N²) is separate from the first term on the right for which the order of the amount of calculations is O(N). By adopting a circuit structure in which the part of the order O(N) of Equation (2) is calculated by software and in which the part of order O(N²) of Equation (2) is calculated by adding or subtracting the sum total of the forces in image cells indicated by lattice vector n depending on the values of lattice vectors n that indicate the position of image cells with respect to the basic cell, virial calculation can be realized by only addition and subtraction, whereby a reduction of the amount of hardware can be realized and a decrease of power consumption can be achieved.

$${V}_{xx} = {\sum }_{i} {x}_{i} {F}_{xi} - \frac{1}{2} {L}_{x} {\sum }_{i} {\sum }_{n} {n}_{x} {\sum }_{j} {f}_{xijn} \qquad ( 2 )$$

The many-body problem calculation device according to the present invention realizes a virial calculation circuit used in molecular dynamics calculations as a virial calculation circuit that does not require multipliers by the incorporation of a means for holding the value of a lattice vector that represents the position coordinates of image cells in periodic boundary conditions and by incorporation of a means for adding or subtracting the sum total of forces in image cells according to the values of lattice vectors. This virial calculation circuit is directed toward reducing the amount of hardware, and, by constituting the hardware of virial calculations by only adder circuits that are controlled by values of lattice vectors, this virial calculation has the effect of enabling a reduction of the amount of hardware and a decrease of power consumption.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

What is claimed is:

1. A two-body interaction calculation circuit comprising:
a center particle coordinate holding unit for holding coordinates of center particles i;
a basic cell information holding unit for holding the size of a basic cell;
a lattice vector holding unit for holding lattice vectors;
a coordinate difference calculation unit for receiving as input coordinates of center particles i that are held by said center particle coordinate holding unit, the size of a basic cell that is held by said basic cell information holding unit, lattice vectors that are held by said lattice vector holding unit, and coordinates of particles j, and for calculating the difference between coordinates of center particles i and coordinates in which coordinates of particles j have been converted to mirror particles;
a distance-squaring calculation unit for calculating the square of distance from differences found in said coordinate difference calculation unit;
a function calculation unit for calculating a function of interaction according to distance based on the square of distance found in said distance-squaring calculation unit;
a force calculation unit for calculating force from the difference found in said coordinate difference delay unit and the function of interaction found in said function calculation unit;
a sum-total calculation unit for calculating a sum total of force for particles i based on force found in said force calculation unit; and
a virial calculation unit for calculating virials based on the sum total of force found in said sum-total calculation unit.

2. A two-body interaction calculation circuit according to claim 1, wherein said force calculation unit is a multiplier that multiplies a difference supplied as output from said coordinate difference delay unit and an interaction function supplied as output from said function calculation unit.

3. A many-body problem calculation device comprising:
a two-body interaction calculation circuit according to claim 1; and
a computer system for obtaining the sum total of force for each particle i and the sum total of virials for all particles i from said two-body interaction calculation circuit and for calculating the entire virial.

4. A many-body problem calculation device comprising:
a two-body interaction calculation circuit according to claim 2; and
a computer system for obtaining the sum total of force for each particle i and the sum total of virials for all particles i from said two-body interaction calculation circuit and for calculating the entire virial.
